(19) United States
(12) Patent Application Publication
(10) Pub. No.: US 2012/0311881 A1
(43) Pub. Date: Dec. 13, 2012

(54) DRYING PAD

(76) Inventor: Pi-Fen LIN, Taipei City (TW)

(21) Appl. No.: 13/592,200

(22) Filed: Aug. 22, 2012

Related U.S. Application Data
(63) Continuation-in-part of application No. 12/571,678, filed on Oct. 1, 2009.

(51) Int. Cl. F26B 19/00

(52) U.S. Cl. 34/90

(57) ABSTRACT

A drying pad includes a flexibly bendable pad body, an air-blowing device and an ultraviolet sterilization device. The pad body is sheet-shaped. A supporting blower is disposed on a bottom-layer sheet of the pad body. An air-blowing device is provided one side of the pad body and communicated with the air-blowing device. The pad body has a plurality of outlet-air vents on a top surface thereof. The supporting blower has a plurality of inlet pipes communicated with the air-blowing device, round casings, and joining pipes. Each round casing has an outlet hole formed on a top thereof. Channels are defined in the supporting blower for guiding the air from the air-blowing device to the outlet holes through the outlet hole. The present invention is capable of blowing air, drying and sterilizing the feet by ultraviolet lights, so that the feet can be dried sufficiently.

### CROSS-REFERENCE TO RELATED APPLICATIONS

This application is a Continuation-in-Part of co-pending application Ser. No. 12/571,678, filed Oct. 1, 2009, for which priority is claimed under 35 U.S.C. §120, the entire contents of which are hereby incorporated by reference.

### BACKGROUND OF THE INVENTION

1. Field of the Invention

The present invention relates to a pad, and in particular to a drying pad having drying and sterilizing functions.

2. Description of Related Arts

In some public places such as swimming pools, rooms for sauna or hot spring, or at home, people often dry their hairs only, and wipe their feet by a towel without drying their feet sufficiently. If people put on socks and shoes with their feet still a little wet, people may feel their feet uncomfortable. Even, the skin of feet may suffer from skin lesion. Thus, sometimes, people use a hair drier to dry their feet. However, such an action is not in good taste and time-wasting without a sufficient drying effect.

Various foot-drying devices are available in the market. For example, Taiwan Patent No. M347915 discloses a foot-drying device, in which a casing is provided with two foot-receiving regions, a fan, and an ultraviolet sterilizing lamp. By this device, the feet of a user can be dried and sterilized by the ultraviolet lights. However, such a device is made to have a hollow casing for receiving the feet of the user, which makes this device bulky, space-occupying, and inconvenient in use.

In order to overcome the above problems, the present Inventor proposes a novel and reasonable structure based on his delicate researches and expert experiences.

### SUMMARY OF THE INVENTION

The objective of the present invention is to provide a drying pad, which is capable of blowing air, drying and sterilizing the feet by ultraviolet lights. By this arrangement, the feet can be dried sufficiently. Thus, the user will not feel his/her feet uncomfortable or the skin of feet may not suffer from skin lesion. Further, the operation of the present invention is easy and time-saving.

Another objective of the present invention is to provide a drying pad, which is compact in size and easy to transport. The user only needs to put his/her feet on the top surface of the pad for use, so that its operation is very convenient.

Another objective of the present invention is to provide a drying pad, which can also act as a hand drier.

In order to achieve the above objectives, the present invention provides a drying pad, which includes: a pad body formed into a sheet and being flexible, the pad body being provided with an inlet-air vent, a top surface of the pad body being provided with a plurality of outlet-air vents, a plurality of channels being connected between the inlet-air vent and the respective outlet-air vents; an air-blowing device provided on the pad body and connected to the inlet-air vent of the pad body; and an ultraviolet sterilization device provided on the pad body.

The present invention further provides a drying pad, which includes: a pad body formed into a sheet, the pad body being provided with an inlet-air vent, a top surface of the pad body being provided with a plurality of outlet-air vents, a plurality of channels being connected between the inlet-air vent and the respective outlet-air vents; an air-blowing device connected to the inlet-air vent of the pad body; and an ultraviolet sterilization device provided on the pad body

The present invention has advantageous features as follows: the drying pad of the present invention can be put in front of the door of a bathroom to act as a mat for blowing air, drying and ultraviolet sterilizing the feet of the user. In this way, the feet can be dried sufficiently. Thus, the user will not feel his/her feet uncomfortable, and the skin of feet may not suffer from skin lesion. Therefore, the operation of the present invention is easy and time-saving.

The main body of the drying pad of the present invention is a sheet-like pad body, which is compact in size and easy to transport. The user only needs to put his/her feet on the top surface of the pad body for use, so that its operation is very convenient.

Further, the pad body of the present invention is made of flexible materials, so that it can be bent and disposed in a seat. In this way, the present invention also acts as a hand drier for drying and sterilizing the hands, which makes the present invention to have a further purpose.

In order to further understand the characteristics and technical contents of the present invention, a description relating thereto will be made with reference to the accompanying drawings. However, the drawings are illustrative only but not used to limit the present invention.

### BRIEF DESCRIPTION OF THE DRAWINGS

FIG. 1 is a perspective view showing the drying pad according to the first embodiment of the present invention;

FIG. 2 is a cross-sectional view showing the drying pad according to the first embodiment of the present invention;

FIG. 3 is a schematic view showing the operating state of the drying pad according to the first embodiment of the present invention;

FIG. 4 is a perspective view showing the drying pad according to the second embodiment of the present invention;

FIG. 5 is a perspective view showing the drying pad according to the third embodiment of the present invention;

FIG. 6 is a plan view showing the drying pad according to the fourth embodiment of the present invention;

FIG. 7 is a perspective view showing the drying pad according to the fifth embodiment of the present invention;

FIG. 8 is a perspective view showing the drying pad according to the sixth embodiment of the present invention;

FIG. 9 is a perspective view showing the drying pad according to the seventh embodiment of the present invention; and

FIG. 10 is a cross-sectional view showing the drying pad according to the eighth embodiment of the present invention.

FIG. 11 is a perspective view showing a supporting blower with an air-blowing device according to the present invention.

FIG. 12 is a cross-sectional view showing the drying pad according to the ninth embodiment of the present invention.

### DETAILED DESCRIPTION OF THE PREFERRED EMBODIMENTS

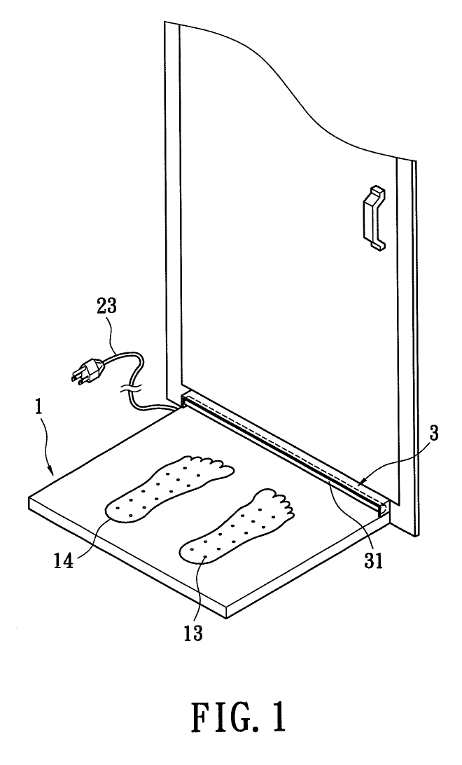

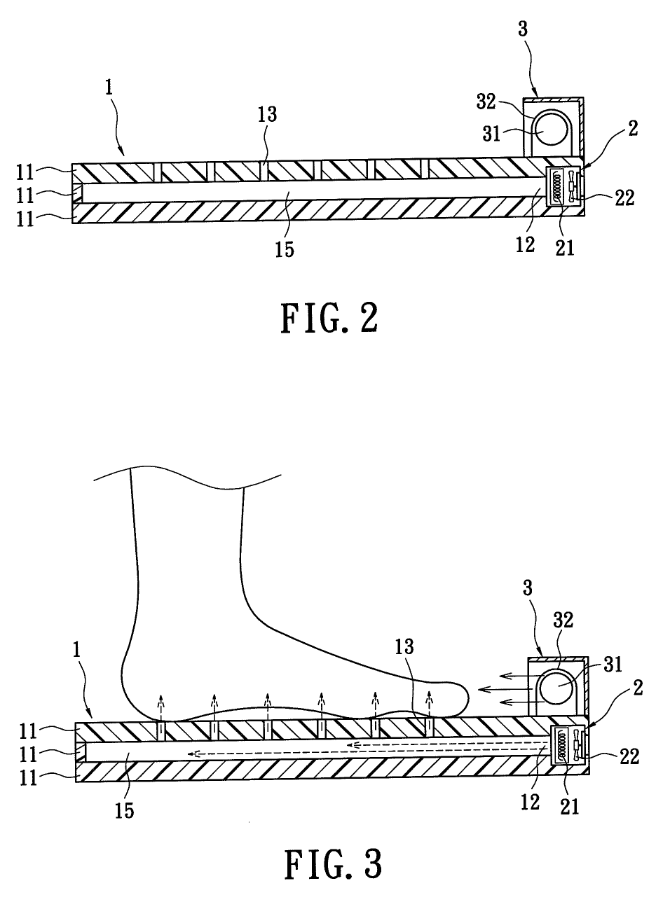

Please refer to FIGS. 1 to 3. The present invention provides a drying pad, which includes a pad body 1, an air-blowing device 2, and an ultraviolet sterilization device 3. The pad body 1 is formed into a sheet and is made of flexible materials, so that the pad body 1 can be bent flexibly. The shape and size of the pad body 1 are not limited to any specific forms, and can be changed according to practical demands. In the present embodiment, the pad body 1 is formed into a rectangular shape. The pad body 1 can be integrally formed into one piece, or it can be constituted of a plurality of overlapped sheets 11. The front side of the pad body 1 is provided with an inlet-air vent 12. However, the location of the inlet-air vent 12 is not limited to the front side of the pad body 1 only, and it can be provided at the rear side, left side, right side of the pad body 1 or other locations. The top surface of the pad body 1 is provided with a plurality of outlet-air vents 13. In the present embodiment, the top surface of the pad body 1 is formed with two foot-receiving regions 14 for allowing the feet of a user to be put therein. The outlet-air vents 13 and the two foot-receiving regions 14 are provided on the upper-layer sheet 11. The outlet-air vents 13 are arranged in the two foot-receiving regions 14. A plurality of channels 15 is connected between the inlet-air vent 12 and the respective outlet-air vents 13. The channels 15 are provided on the middle-layer sheet 11. The channels 15 are used to communicate the inlet-air vent 12 with the outlet-air vents 13.

The air-blowing device 2 is provided on the pad body 1. In the present embodiment, the air-blowing device 2 is provided on the front side of the pad body 1. The air-blowing device 2 is connected with the inlet-air vent 12 of the pad body 1. However, the location of the air-blowing device 2 is not limited to this, and the air-blowing device 2 can be provided in the rear side, left side, right side of the pad body 1 or other locations. In the present embodiment, the air-blowing device 2 is an electric heating air-blowing device, which includes a heating unit 21 and an air-blowing unit 22. In the present embodiment, the heating unit 21 is an electric heater, and the air-blowing unit 22 is a fan. The heating unit 21 and the air-blowing unit 22 are electrically connected to power lines 23, whereby necessary electricity can be delivered to the heating unit 21 and the air-blowing unit 22 for driving the heating unit 21 and the air-blowing unit 22 to operate. In this way, the heating unit 21 can be activated to raise its temperature. The outside air is drawn by the air-blowing unit 22 to be heated by the heating unit 21, thereby generating high-temperature air. The high-temperature air flows through the inlet-air vent 12, the channels 15, and the outlet-air vents 13 of the pad body 1 to dry the feet put on the pad body 1 (FIG. 3).

The above-mentioned air-blowing device 2 may be a cold air blowing device, in which only an air-blowing unit 22 is provided but no heating unit. The outside air is drawn by the air-blowing device 2 to flow through the inlet-air vent 12, the channels 15, and the outlet-air vents 13 of the pad body 1 to dry the feet of the user put on the pad body 1 with cold air.

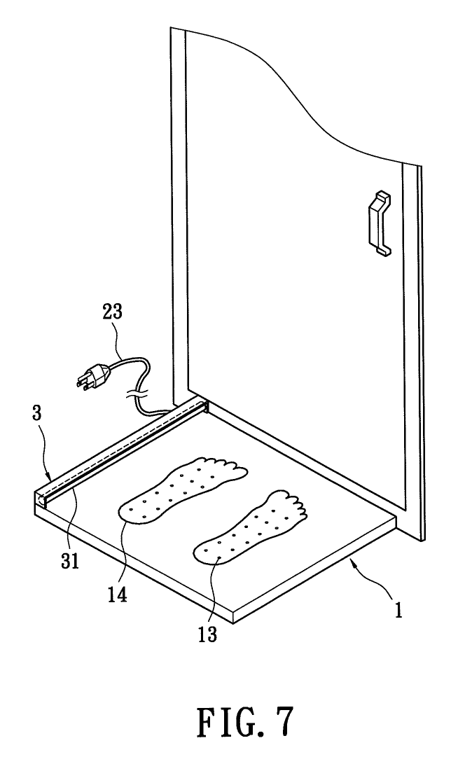

The ultraviolet sterilization device 3 is provided on the pad body 1. In the present embodiment, the ultraviolet sterilization device 3 is provided at the front side of the pad body 1 to be in front of the two foot-receiving regions 14. However, the location of the ultraviolet sterilization device 3 is not limited thereto, and it can be provided at the rear side, left side (FIG. 7), right side of the pad body 1 or other locations. The ultraviolet sterilization device 3 has at least one ultraviolet lamp 31. Both ends of the ultraviolet lamp 31 are inserted into two lamp sockets 32 respectively. The two lamp sockets 32 are electrically connected to the power line 23, so that necessary electricity can be delivered to the ultraviolet lamp 31 of the ultraviolet sterilization device 3 to light it up. Thus, the ultraviolet sterilization device 3 can generate ultraviolet lights to sterilize the feet of the user put on the pad body 1 (FIG. 3). With the above structure, the drying pad of the present invention can be formed.

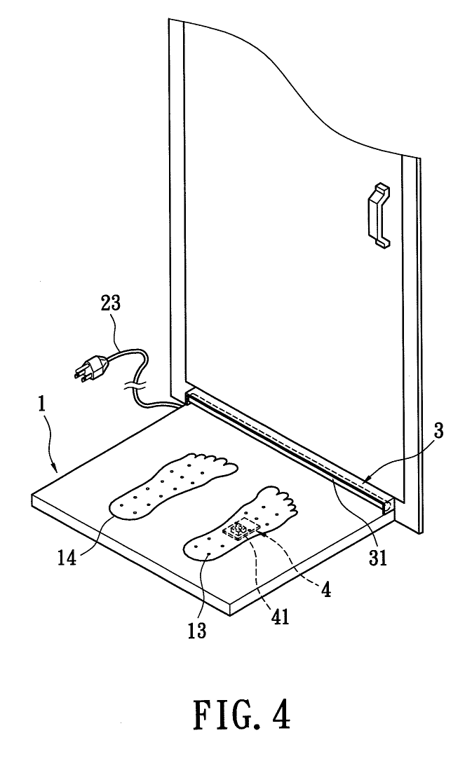

Please refer to FIG. 4. In the present embodiment, the pad body 1 is provided with a circuit board 4. The circuit board 4 is electrically connected with a sensor 41. The air-blowing device 2 and the ultraviolet sterilization device 3 are electrically connected to the circuit board 4. When the feet of the user are put on the pad body 1, the sensor 41 can sense the feet to automatically activate the air-blowing device 2 and the ultraviolet sterilization device 3 for operation.

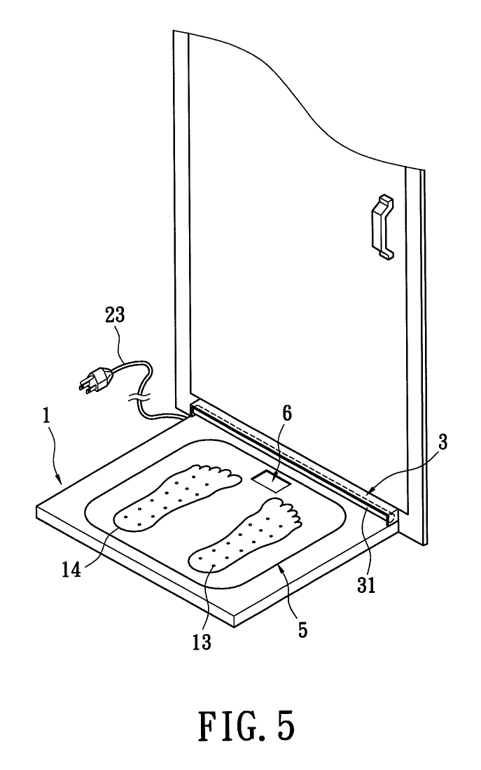

Please refer to FIG. 5. In the present embodiment, the pad body 1 is provided with a plate scale 5. The plate scale 5 may be an electronic scale or a mechanical scale. The two foot-receiving regions 14 are provided on the plate scale 5. Further, the pad body 1 is provided with a display 6 which may be a liquid crystal display. When the feet of the user are put on the pad body 1, the user can measure his/her weight by means of the plate scale 5 and the measured value can be displayed on the display 6. With this structure, the drying pad also acts as a weight scale.

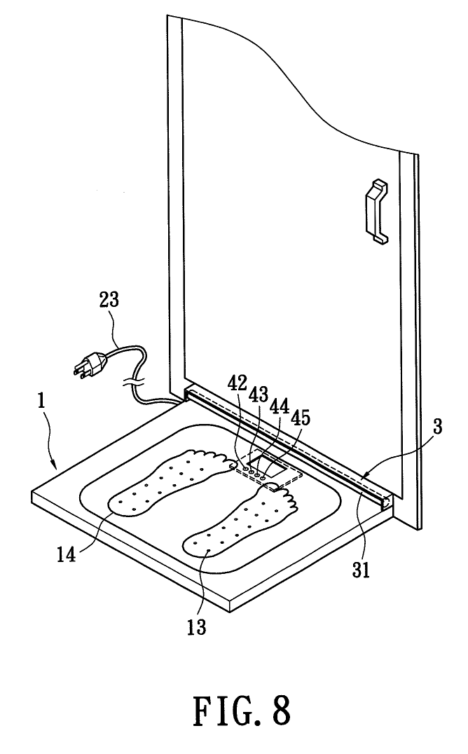

Further, in the present invention, the circuit board 4 is electrically connected with a music device 42, a temperature-adjusting device 43, an air speed adjusting device 44, and a time-adjusting device 45 (FIG. 8). With this arrangement, the present invention has a plurality of additional functions such as playing music, adjusting the temperature and air speed of the air-blowing device 2, setting and adjusting time of the air-blowing device 2 and the ultraviolet sterilization device 3. Since the above-mentioned auxiliary devices 42, 43, 44 and 44 are well known, the description thereof is omitted for clarity.

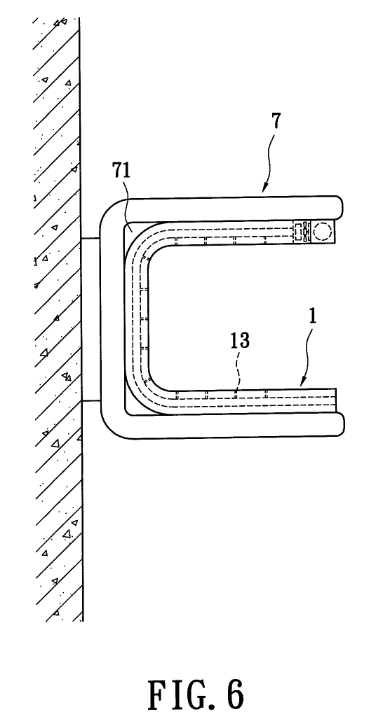

Further, please refer to FIG. 6. Since the pad body 1 is made of flexible materials, it can be bent flexibly. After bending, the pad body 1 can be disposed in a seat 7 fixed on the wall. The interior of the seat 7 is formed with an accommodating space 71. The front side of the accommodating space 71 is formed into an opening, so that the pad body 1 after bending can be disposed therein with the outlet-air vents 13 of the pad body 1 facing toward the front side of the accommodating space 71. With this structure, the present invention acts as a heating device having drying and sterilizing functions, and for example, it can be used as a hand drier.

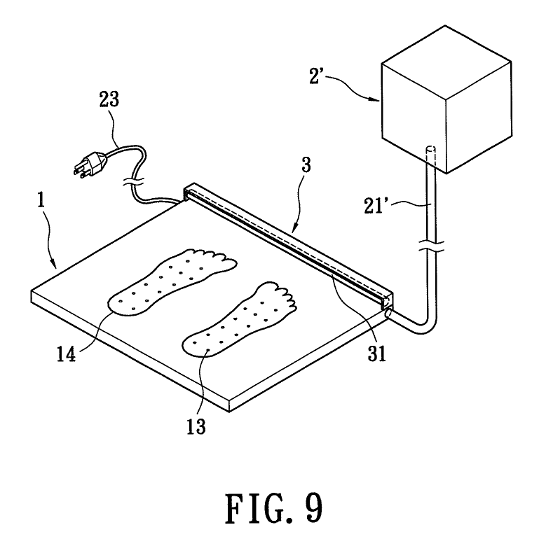

Please refer to FIG. 9. The air-blowing device 2 of the present invention can be omitted and using an external air-blowing device 2′ instead. The air-blowing device 2′ may be a hand drier available in the market. The inlet-air vent 12 of the pad body 1 is connected to the air-blowing device 2′ with a connecting pipe 21′. In this way, the high-temperature air can be delivered through the connecting pipe 21′ to the outlet-air vents 13, so as to dry the feet of the user put on the pad body 1.

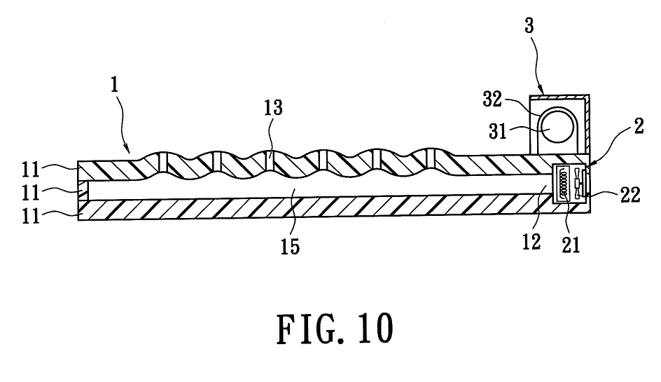

Please refer to FIG. 10. The top surface of the pad body 1 may be formed into a waved shape, thereby facilitating the drying effect.

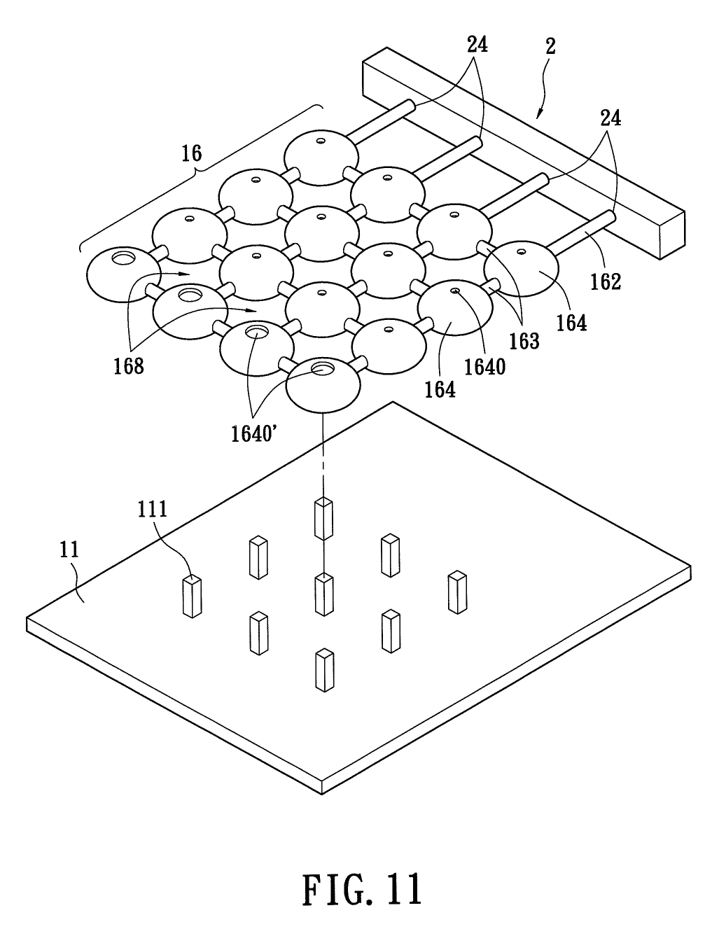

Please refer to FIG. 11. For providing a bendable structure to strongly support the user's weight on the drying pad, the present invention further provides a mesh-shaped supporting blower 16 disposed on the bottom-layer sheet 11. The supporting blower 16 includes a plurality of inlet pipes 162, round casings 164, and joining pipes 166. The air-blowing device 2 provides a plurality of outlet openings 24 to connect the inlet pipes 162 respectively. The other end of each inlet pipe 162 is connected to one round casing 164. The round casings 164 are arranged in grid manner and connected through the joining pipes 166. Further, there is a plurality of mesh holes 168 formed among the round casings 164. Each round casing 164 has an outlet hole 1640 formed on a top thereof for exhausting air. The outlet holes can has different apertures, as shown in the outlet hole 1640 or 1640′. In this embodiment, the joining pipes 166 preferably are made of bendable material. Therefore, the round casings 164 can be rolled row by row toward the air-blowing device 2.

In this embodiment, the drying pad 10 further has a plurality of supporting rods 111 fixed on the bottom-layer sheet 11. The supporting rods 111 are disposed in the mesh holes 168 respectively for providing a stronger supporting ability. The supporting rods 111 can be made of elastic material, such as rubber, or plastic.

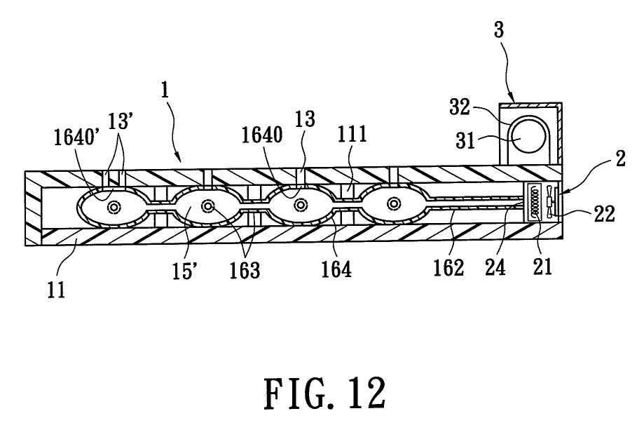

Please refer to FIG. 12. The supporting blower 16 is disposed between the upper-layer sheet 11 and the bottom-layer sheet 11. The supporting rods 111 also support the upper-layer sheet 11. In this embodiment, the outlet-air vents 13 are aligned with the outlet holes 1640 of the round casings 164. In an alternative embodiment, the aperture of outlet hole 1640′ can be larger than that of the outlet-air vent 13′, and each outlet hole 1640′ can be communicated with many outlet-air vents 13′. Therefore, even some of the outlet-air vents 13′ are closed by user's feet, the air still can be blew out from the outlet hole 1640′ to other outlet-air vents 13′. In this embodiment, each row of the round casings 164 can form one channel 15′ for guiding the air from the air-blowing device 2 to the outlet holes 1640, 1640′. However, the channels 15′ are also arranged in mesh shape.

The drying pad of the present invention can be put in front of the door of a bathroom to act as a mat for blowing air, drying and sterilizing the feet of the user by ultraviolet lights. Thus, the feet can be dried sufficiently, so that the user may not feel his/her feet uncomfortable, or the skin may not suffer from skin lesion. Further, the operation of the present invention is easy and time-saving.

Moreover, since the main body of the drying pad of the present invention is a sheet-like pad body 1, it is compact in size and easy to transport. The user only needs to put his/her feet on the top surface of the pad body 1 for use. Thus, the present invention is very convenient to use.

Moreover, the pad body 1 of the present invention is made of flexible materials, so that it can be bent flexibly and thus disposed in a seat 7, thereby acting as a hand drier for drying and sterilizing hands of the user. Thus, the present invention has a further function.

The above-mentioned descriptions represent merely the preferred embodiments of the present invention, without any intention to limit the scope of the present invention thereto. Various equivalent changes, alternations or modifications based on the claims of present invention are all consequently viewed as being embraced by the scope of the present invention.

What is claimed is:

1. A drying pad, comprising:
a flexibly bendable pad body formed into the shape of a sheet, the pad body having a bottom-layer sheet;
an air-blowing device provided on one side of the pad body; and
a mesh-shaped supporting blower disposed on the bottom-layer sheet of the pad body, the supporting blower having a plurality of inlet pipes communicated with the air-blowing device, a plurality of round casings, and a plurality of joining pipes, wherein the round casings are communicated by the joining pipes, each round casing having at least one outlet hole formed on a top thereof for exhausting air;
wherein the supporting blower has a plurality of channels defined therein in a mesh-shaped manner transversing substantially the entire length of the pad body.

2. The drying pad according to claim 1, wherein the pad body is integrally formed in one piece or constituted of a plurality of overlapped sheets.

3. The drying pad according to claim 1, wherein the pad body further has an upper-layer sheet disposed above the bottom-layer sheet and supported by the supporting blower, the upper-layer sheet is formed with two foot-receiving regions on a top surface thereof and a plurality of outlet-air vents arranged in the two foot-receiving regions.

4. The drying pad according to claim 3, wherein the outlet-air vents are aligned with the outlet holes of the round casings.

5. The drying pad according to claim 3, wherein the aperture of outlet hole is larger than that of the outlet-air vent, each outlet hole is communicated with a plurality of the outlet-air vents.

6. The drying pad according to claim 1, wherein the air-blowing device is an electric heating air-blowing device, the air-blowing device comprises a heating unit and an air-blowing unit, the heating unit is an electric heater, and the air-blowing unit is a fan.

7. The drying pad according to claim 1, wherein the air-blowing device is a cold air blowing device.

8. The drying pad according to claim 1, further comprising an ultraviolet sterilization device provided on the pad body, wherein the air-blowing device and the ultraviolet sterilization device are electrically connected to a power line respectively.

9. The drying pad according to claim 1, wherein the ultraviolet sterilization device is provided on one side of the pad body.

10. The drying pad according to claim 1, wherein a circuit board is provided on the pad body.

11. The drying pad according to claim 10, wherein the circuit board is electrically connected to a sensor.

12. The drying pad according to claim 10, wherein a plate scale and a display are provided on the pad body.

13. The drying pad according to claim 10, wherein the circuit board is electrically connected with a music device.

14. The drying pad according to claim 10, wherein the circuit board is electrically connected with a temperature-adjusting device and an air speed adjusting device.

15. The drying pad according to claim 10, wherein the circuit board is electrically connected with a time-adjusting device.

16. The drying pad according to claim 1, further comprising a seat, the interior of the seat being formed with an accommodating space, the pad body being bent to be put in the accommodating space of the seat.

17. The drying pad according to claim 1, wherein the top surface of the pad body is formed into a waved shape.

18. The drying pad according to claim 1, wherein the supporting blower defines a plurality of mesh holes among the round casings, wherein the drying pad further has a plurality of supporting rods fixed on the bottom-layer sheet disposed in the mesh holes respectively.

19. The drying pad according to claim 18, wherein the supporting rods are made of made of elastic material.
